(19) United States
(12) Patent Application Publication
(10) Pub. No.: US 2026/0083373 A1
(43) Pub. Date: Mar. 26, 2026

(54) METHOD FOR PRODUCING A BI- OR MULTIPOLAR LEAD

(71) Applicant: Heraeus Medevio GmbH & Co. KG, Hanau (DE)

(72) Inventors: Leoni WILHELM, Hanau (DE); Ilias NIKOLAIDIS, Hanau (DE)

(21) Appl. No.: 19/332,306

(22) Filed: Sep. 18, 2025

(30) Foreign Application Priority Data
Sep. 23, 2024 (DE) 102024127317.8

(51) Int. Cl. A61B 5/273

(52) U.S. Cl. CPC A61B 5/273; A61B 2562/125

(57) ABSTRACT

A method for producing a bi- or multipolar lead for a medical device, comprising the method steps of providing a cable comprising an outer insulation, an inner lumen, wherein the inner lumen is arranged coaxially with respect to the outer insulation and extends in a longitudinal direction from a proximal end to a distal end of the cable, and at least one electrically conductive channel, wherein the at least one electrically conductive channel is arranged between the outer insulation and the inner lumen of the cable, and wherein the at least one electrically conductive channel is formed by at least one insulated conductor which comprises an electrical conductor and an insulating layer.

### CROSS-REFERENCE TO RELATED APPLICATION

This application claims priority pursuant to 35 U.S.C. 119 (a) to German Application No. 102024127317.8, filed Sep. 23, 2024, which application is incorporated herein by reference in its entirety.

### FIELD OF THE INVENTION

A bi- or multipolar lead is suitable for a medical device and, in one embodiment, for a temporary medical measuring lead. However, the bi- or multipolar lead can also be used in other technical areas.

### BACKGROUND

Bi- or multipolar leads, and especially multipolar leads, for medical devices are usually very complex to produce since their production requires a large number of different components which must be assembled in a multitude of individual steps. The production of small-format leads, which are particularly in demand in the field of medical devices, is even more complex. The complex design of bi- or multipolar leads and especially their production usually led to high prices for the final product.

The high prices of known bi- or multipolar leads are particularly problematic when the leads are to be used in medical devices for a relatively short period of time, e.g., in a temporary device for medical tests. To conduct temporary medical tests in an economically viable manner, cheaper unipolar leads are therefore chosen for the tests. However, the use of a unipolar lead may limit the success of the test, which may result in fewer patients receiving appropriate medical treatment in the form of a permanent medical device.

In addition, a bi- or multipolar lead designed and produced using a large number of individual components may demonstrate lower reliability because there are more possible failure modes of the individual parts. Thus, lower reliability may lead to a higher risk of failure of the lead, which in turn may have significant consequences, such as for a patient's health, if the lead is used in a medical device.

In particular, the electrical connection of electrodes to the lead is often complex to do in practice and carries a high risk of failure.

### SUMMARY

In view of the above, it is desirable to provide a method for producing a bi- or multipolar lead which is simple, reliable, flexible and efficient to carry out. In particular, it is desirable to simplify the electrical contact of the electrodes of the lead and design it in such a way that the risk of failure of the contacted electrodes is as low as possible. Furthermore, the method should be carried out as mechanically as possible, i.e. with as few manual steps as possible.

The invention relates to a method for producing a bi- or multipolar lead for a medical device, comprising the method steps of:
a) providing a cable comprising
   an outer insulation,
   an inner lumen, wherein the inner lumen is arranged coaxially with respect to the outer insulation and extends in a longitudinal direction from a proximal end to a distal end of the cable, and,
   at least one electrically conductive channel, wherein the at least one electrically conductive channel is arranged between the outer insulation and the inner lumen of the cable, and, wherein the at least one electrically conductive channel is formed by at least one insulated conductor which comprises an electrical conductor and an insulating layer;
b) providing at least one conductive paste;
c) creating at least one contact opening in the proximity of the distal end of the cable, wherein, in order to create the contact opening, parts of the outer insulation are removed and parts of the insulating layer of at least one of the electrically conductive channels are removed so that the at least one electrically conductive channel can be selectively electrically contacted via the contact opening, wherein the contact opening comprises a contact opening cross-sectional area;
d) filling the contact opening with the conductive paste, wherein a filled conductive paste volume of the conductive paste comprises 105% to 200%, preferably 105% to 150%, more preferably 105% to 120%, of a capacity volume of the contact opening in order to create a projection of the conductive paste protruding from the contact opening;
e) providing at least one electrode, wherein the electrode comprises a contact surface with a contact cross-sectional area, wherein the contact cross-sectional area is larger than the contact opening cross-sectional area;
f) positioning the electrode at the position of the contact opening, wherein the contact surface is oriented in the direction of the contact opening;
g) moving the positioned electrode in the direction of the contact opening so that the projection of the conductive paste is introduced during the movement between the outer insulation and a part of the contact surface which projects beyond the contact opening;
h) curing the conductive paste to fix the electrode in an electrically conductive manner to the electrically conductive channel.

One object of the present invention is to overcome, at least in part, one or more of the disadvantages resulting from the prior art.

It is a further object of the invention to provide a method for producing a bi- or multipolar lead which is as simple, reliable, flexible and cost-effective as possible. In particular, it is an object of the invention to design electrical contacting of electrodes for such a method as simply, reliably, flexibly, cost-effectively and with as few manual method steps as possible. In addition, the electrodes that are electrically contacted in this way should be fixed to the rest of the lead as fail-safe as possible.

A contribution to the at least partial fulfillment of at least one of the aforementioned objects is made by the features of the independent claims. The dependent claims provide preferred embodiments that contribute to the at least partial fulfillment of at least one of the objects.

A first embodiment of the invention is a method for producing a bi- or multipolar lead for a medical device comprising the method steps of:
a) providing a cable comprising
   an outer insulation,
   an inner lumen,
   wherein the inner lumen is arranged coaxially with respect to the outer insulation and extends in a longitudinal direction from a proximal end to a distal end of the cable, and
   at least one electrically conductive channel,
   wherein the at least one electrically conductive channel is arranged between the outer insulation and the inner lumen of the cable, and
   wherein the at least one electrically conductive channel is formed by at least one insulated conductor which comprises an electrical conductor and an insulating layer;
b) providing at least one conductive paste;
c) creating at least one contact opening in the proximity of the distal end of the cable, wherein, in order to create the contact opening, parts of the outer insulation are removed and parts of the insulating layer of at least one of the electrically conductive channels are removed so that the at least one electrically conductive channel can be selectively electrically contacted via the contact opening, wherein the contact opening comprises a contact opening cross-sectional area;
d) filling the contact opening with the conductive paste, wherein a filled conductive paste volume of the conductive paste comprises 105% to 200%, preferably 105% to 150%, more preferably 105% to 120%, of a capacity volume of the contact opening in order to create a projection of the conductive paste protruding from the contact opening;
e) providing at least one electrode, wherein the electrode comprises a contact surface with a contact cross-sectional area, wherein the contact cross-sectional area is larger than the contact opening cross-sectional area;
f) positioning the electrode at the position of the contact opening, wherein the contact surface is oriented in the direction of the contact opening;
g) moving the positioned electrode in the direction of the contact opening so that the projection of the conductive paste is introduced during the movement between the outer insulation and a part of the contact surface which projects beyond the contact opening;
h) curing the conductive paste to fix the electrode in an electrically conductive manner to the electrically conductive channel.

In a preferred embodiment of the method, the conductive paste comprises a conductive paste composition which contains a proportion of at most 5 percent by weight of volatile organic substances, based on the total mass of the conductive paste composition. This embodiment is a second embodiment of the invention, which is preferably dependent on the first embodiment of the invention.

In a preferred embodiment of the method, the volatile organic substances are selected from the group of solvents, preferably organic solvents, such as mono[(C12-14-alkyloxy)methyl] derivatives, ethyl acetate, tetrahydrofuran and neopentyl glycol diglycidyl ether. This embodiment is a third embodiment of the invention, which is preferably dependent on the second embodiment of the invention.

In a preferred embodiment of the method, the conductive paste composition contains a proportion of at least 0.5 percent by weight of volatile organic substances, based on the total mass of the conductive paste composition. This embodiment is a fourth embodiment of the invention, which is preferably dependent on the second or third embodiment of the invention.

In a preferred embodiment of the method, the conductive paste composition contains a proportion of 70-90 percent by weight of conductive particles, in particular electrically conductive particles. This embodiment is a fifth embodiment of the invention, which is preferably dependent on the second to fourth embodiment of the invention.

In a preferred embodiment of the method, the particles, in particular the electrically conductive particles, are metal particles. This embodiment is a sixth embodiment of the invention, which is preferably dependent on the fifth embodiment of the invention.

In a preferred embodiment of the method, the metal particles comprise a metal which is selected from the group consisting of platinum, iridium, tantalum, palladium, titanium, iron, gold, molybdenum, niobium, tungsten, nickel, chromium, cobalt, stainless steel, nitinol, alloys of any of these metals, and composites of any of these metals. This embodiment is a seventh embodiment of the invention, which is preferably dependent on the sixth embodiment of the invention.

In a preferred embodiment of the method, the conductive paste composition comprises a polymer which is selected from the group consisting of polyimides and polyurethanes. This embodiment is an eighth embodiment of the invention, which is preferably dependent on the second to seventh embodiment of the invention.

In a preferred embodiment of the method, the projection in the radial direction has a height in a range of 0.1 mm to 0.5 mm. This embodiment is a ninth embodiment of the invention, which is preferably dependent on one of the preceding embodiments of the invention.

In a preferred embodiment of the method, the curing in method step h) causes a radial contraction, in particular at least a radial contraction, of the conductive paste which corresponds to a maximum of 5% of a radial height of the contact opening. This embodiment is a tenth embodiment of the invention, which is preferably dependent on one of the preceding embodiments of the invention.

In a preferred embodiment of the method, the electrode is a ring electrode. This embodiment is an eleventh embodiment of the invention, which preferably depends upon one of the preceding embodiments of the invention.

In a preferred embodiment of the method, the movement in method step g) comprises a radial compression of the ring electrodes. This embodiment is a twelfth embodiment of the invention, which is preferably dependent on the eleventh embodiment of the invention.

In a preferred embodiment of the method, the positioning in method step f) comprises drawing, or in other words pushing, the ring electrode onto the cable. This embodiment is a thirteenth embodiment of the invention, which is preferably dependent on the eleventh or twelfth embodiment of the invention.

In a preferred embodiment of the method, the contact surface is a radially inner ring surface of the ring electrode. This embodiment is the fourteenth embodiment of the invention, which is preferably dependent on the eleventh to thirteenth embodiments of the invention.

In a preferred embodiment of the process, the curing in method step h) is carried out at a temperature which lies in a range between 110° C. and 140° C. This embodiment is a fifteenth embodiment of the invention, which preferably depends upon one of the preceding embodiments of the invention.

With respect to the embodiments described herein, the elements of which “have,” or “comprise,” a particular feature (for example, a material), in principle, a further embodiment is always contemplated in which the relevant element consists solely of the feature, i.e., does not comprise any other constituents. The word “comprise” or “comprising” is used herein synonymously with the word “have” or “having.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products and devices described herein, and vice versa.

All such considered combinations are not explicitly listed in all instances, simply in order to keep the description brief. Technical solutions known to be equivalent to the features described herein are also intended in principle to be encompassed by the scope of the invention.

In the present description, specifications of ranges also contain the values specified as limits. A specification of the type “in the range from X to Y” with respect to a quantity A consequently means that A can take the values X, Y and values between X and Y. Ranges which are limited on one side, of the type “up to Y” for a size A, accordingly mean a value Y and less than Y.

Some of the features described are associated with the term “substantially”. The term “substantially” is to be understood in such a way that, under real conditions and manufacturing techniques, a mathematically exact interpretation of terms such as “superimposition”, “perpendicular”, “diameter” or “parallelism” can never be given exactly, but only within certain manufacturing error tolerances. For example, “substantially perpendicular axes” enclose an angle of 85 degrees to 95 degrees relative to one another, and “substantially equal volumes” comprise a variation of up to 5% by volume. For example, a “device consisting substantially of plastics” comprises a plastics content of ≥95 to ≤100% by weight. For example, a “substantially complete filling of a volume B” comprises a filling of ≥95 to ≤100% by volume of the total volume of B.

When an indefinite or definite article is used when referring to a singular noun, such as “a”, “an” or “the”, it includes a plural of that noun unless explicitly stated otherwise. When the term “comprising” is used in the present description and claims, other elements are not thereby excluded.

For the purposes of the present invention, the terms “consisting substantially of” and “consisting of” are considered embodiments of the term “comprising”. When a group is defined below as comprising at least a certain number of embodiments, this is also to be understood as disclosing a group which, in one embodiment, consists substantially only of these embodiments or, in one embodiment, consists only of these embodiments.

Terms such as “obtainable” or “definable” and “obtained” or “defined” are used interchangeably. This means, for example, that the term “obtained” does not imply that an embodiment must be obtained, for example, by the sequence of steps following the term “obtained”, unless the context clearly dictates otherwise, although such a limited understanding is always included in the terms “obtained” or “defined” as an embodiment. Whenever the terms “including” or “with” are used, these terms are synonymous with “comprising” as defined above.

### DETAILED DESCRIPTION

A first object of the invention relates to methods for producing a bi- or multipolar lead for a medical device, comprising the method steps of:
a) providing a cable comprising
   an outer insulation,
   an inner lumen,
   wherein the inner lumen is arranged coaxially with respect to the outer insulation and extends in a longitudinal direction from a proximal end to a distal end of the cable, and
   at least one electrically conductive channel,
   wherein the at least one electrically conductive channel is arranged between the outer insulation and the inner lumen of the cable, and
   wherein the at least one electrically conductive channel is formed by at least one insulated conductor which comprises an electrical conductor and an insulating layer;
b) providing at least one conductive paste;
c) creating at least one contact opening in the proximity of the distal end of the cable, wherein, in order to create the contact opening, parts of the outer insulation are removed and parts of the insulating layer of at least one of the electrically conductive channels are removed so that the at least one electrically conductive channel can be selectively electrically contacted via the contact opening, wherein the contact opening comprises a contact opening cross-sectional area;
d) filling the contact opening with the conductive paste, wherein a filled conductive paste volume of the conductive paste comprises 105% to 200%, preferably 105% to 150%, more preferably 105% to 120%, of a capacity volume of the contact opening in order to create a projection of the conductive paste protruding from the contact opening;
e) providing at least one electrode, wherein the electrode comprises a contact surface with a contact cross-sectional area, wherein the contact cross-sectional area is larger than the contact opening cross-sectional area;
f) positioning the electrode at the position of the contact opening, wherein the contact surface is oriented in the direction of the contact opening;
g) moving the positioned electrode in the direction of the contact opening so that the projection of the conductive paste is introduced during the movement between the outer insulation and a part of the contact surface which projects beyond the contact opening;
h) curing the conductive paste to fix the electrode in an electrically conductive manner to the electrically conductive channel.

The method serves to produce a bi- or multipolar lead for a medical device in a simple and efficient manner, wherein the method also makes a large number of different leads, in particular leads provided with a different number of electrodes, easily and efficiently accessible due to its high flexibility. The method enables efficient, simple and cost-effective electrical contacting of the electrodes, particularly in the form of ring electrodes, at any point of the insulated lead cable. The electrodes electrically fixed in this way also have a high degree of reliability. Due to the efficient implementation of the method, leads can also be produced for temporary applications, such as temporary medical measuring arrangements, which would not be attractive from an economic perspective using other methods.

A “bipolar” lead in the sense of the invention is understood to mean a lead comprising two electrodes which are each electrically connected to one or more electrically conductive channels near the distal end of the lead cable. A “multipolar” lead in the sense of the invention is understood to mean a lead comprising at least three electrically conductive channels and three electrodes which are each electrically connected to one of the conductive channels near the distal end of the lead cable. In general, the polarity of the lead is determined by the number of conductive channels in the lead. Since each of the conductive channels is selectively connected to an electrode near the distal end of the lead cable, the number of electrodes also determines the minimum number of electrically conductive channels near the distal end of the lead cable. Thus, a multipolar lead comprising four electrodes comprises a minimum of four electrically conductive channels.

“Near the distal end” of the lead cable means that a position lies within the last 30%, preferably the last 20%, most preferably the last 10% of the lead cable length, and “near the proximal end” means that a position lies within the first 30%, preferably the first 20%, most preferably the first 10% of the lead cable length.

An “electrically conductive channel” within the meaning of the invention is a means for selectively electrically connecting an electrical part (e.g., a ring electrode) near the proximal end of the lead to an electrode, preferably a ring electrode, near the distal end of the lead.

An “inner lumen” in the sense of the invention is a free space in the middle of the bi- or multipolar lead. For example, the inner lumen serves for the use of a guidewire to facilitate insertion of the lead into a patient.

In method step a), a cable is provided. The cable has a proximal end and a distal end. The length of the cable can be selected by a person skilled in the art with regard to the use of the lead according to the invention and considering the present specification. For example, the cable can have a length in the range of 50 to 5000 mm, preferably 100 to 2000 mm and particularly preferably 200 to 1000 mm. In an exemplary embodiment, the cable has a length of approximately 500 mm.

The cable comprises an outer insulation. Preferably, the outer insulation has an outer diameter in a range of 200 to 5000 μm, more preferably 300 to 3000 μm and most preferably 500 to 1500 μm. In an exemplary embodiment, the outer diameter of the outer insulation is about 700 μm. It is understood that the outer diameter of the outer insulation of the cable can also correspond to the outer diameter of the cable. The outer insulation preferably has a wall thickness in the range of 2 to 300 μm, particularly preferably from 5 to 150 μm, and most preferably from 20 to 100 μm. According to a preferred embodiment of the present invention, the outer insulation has an outer diameter in a range from 300 to 3000 μm, particularly preferably from 500 to 1500 μm, and a wall thickness in a range from 5 to 150 μm, particularly preferably from 20 to 100 μm. In an exemplary embodiment, the wall thickness is approximately 60 μm.

Preferably, the outer insulation consists of a polymer that is selected from the group consisting of silicones, polyolefins (e.g., polyethylene), polyurethanes, polyimides, polyamides, polyaryletherketone (e.g., polyetheretherketones), fluorinated polymers (e.g., selected from of ethylenetetrafluoroethylene, polytetrafluoroethylene, perfluoroalkoxyalkanes, polyvinylidene fluorides, fluorinated ethylenepropylene, and mixtures thereof), and mixtures thereof. According to a preferred embodiment, the outer insulation comprises a polymer selected from the group consisting of silicones, polyolefins (e.g., polyethylene), polyurethanes, polyimides, polyamides, polyaryletherketones (e.g., polyetheretherketone), fluorinated polymers (e.g., selected from the group of ethylenetetrafluoroethylene, polytetrafluoroethylene, perfluoroalkoxyalkanes, polyvinylidene fluorides, fluorinated ethylenepropylene, and mixtures thereof) and mixtures thereof, and has an outer diameter in the range of 300 to 3000 μm, and most preferably 500 to 1500 μm, and a wall thickness in the range of 5 to 150 μm, and most preferably 20 to 100 μm.

The cable comprises an inner lumen, wherein the inner lumen is coaxial with the outer insulation and extends in a longitudinal direction from a proximal end to the distal end of the cable. In a radial cross-section of the cable, the inner lumen is therefore located substantially on the longitudinal axis of the cable in its center.

The inner lumen preferably has the dimensions of a stylet, i.e. a slender medical probe or device for implantation of the lead can be positioned and/or inserted into the inner lumen of the lead. Preferably, the inner lumen has a diameter in the range from 10 to 1000 μm, preferably in the range from 50 to 500 μm, particularly preferably in the range from 200 to 400 μm. The diameter of the inner lumen can, for example, be about 350 μm.

The inner lumen can correspond to a hollow strand that is formed only by the boundaries of the at least one conductive channel which is arranged between the outer insulation and the inner lumen of the cable. In one embodiment, the inner lumen is accordingly formed by at least one conductive channel that extends in a longitudinal direction from a proximal end to a distal end of the cable. At least one conductive channel can, for example, be spirally wound to form the inner lumen of the cable. In one embodiment, the cable does not include an inner tube that forms the inner lumen of the cable.

However, it is preferred that the inner lumen of the cable is formed by an inner structure, preferably an inner liner, such as an inner tube or an inner hose, particularly preferably an inner polymer hose. The inner liner can be considered a support structure between at least one conductive channel that is arranged between the outer insulation and the inner lumen of the cable. The inner liner can give the cable improved stability. The inner liner can comprise a flat metal strip which is braided or woven or consist of such a metal strip. Preferably, the flat metal strip is coated with a polymer film such as a polymer film made of Pebax®.

The inner liner can have specific dimensions. The inner liner can have an outer diameter in the range from 10 to 1000 μm, for example in the range from 50 to 500 μm, particularly preferably in the range from 200 to 400 μm. In an exemplary embodiment, the outer diameter of the inner liner is about 350 μm, and the inner diameter of the inner liner is about 250 μm. The inner liner can have a wall thickness in the range of 2 to 200 μm, preferably 10 to 100 μm, and particularly preferably 20 to 80 μm. According to a preferred embodiment of the present invention, the inner liner has an outer diameter in the range from 50 to 500 μm, preferably in the range from 200 to 400 μm, and a wall thickness in the range from 10 to 100 μm, particularly preferably from 20 to 80 μm.

The inner liner in the form of a polymer tube is selected from the group consisting of silicones, polyolefins (e.g., polyethylene), polyurethanes, polyimides, polyamides, polyaryletherketone (e.g., polyetheretherketone), fluorinated polymers (e.g., selected from the group of ethylenetetrafluoroethylene, polytetrafluoroethylene, perfluoroalkoxyalkanes, polyvinylidene fluorides, fluorinated ethylenepropylene, and mixtures thereof), and mixtures thereof. According to a preferred embodiment, the inner liner comprises a polymer selected from the group consisting of silicones, polyolefins (e.g. polyethylene), polyurethanes, polyimides, polyamides, polyaryletherketones (e.g. polyetheretherketone), fluorinated polymers (e.g. selected from the group ethylenetetrafluoroethylene, polytetrafluoroethylene, perfluoroalkoxyalkanes, polyvinylidene fluorides, fluorinated ethylenepropylene and mixtures thereof) and mixtures thereof, and has an outer diameter in the range of 50 to 500 μm, preferably in the range of 200 to 400 μm, and a wall thickness in the range of 10 to 100 μm, preferably 20 to 80 μm.

The cable includes at least one electrically conductive channel. For example, the cable can have between one and eighteen electrically conductive channels. The at least one electrically conductive channel is arranged between the outer insulation and the inner lumen of the cable.

Preferably, the cable comprises at least four electrically conductive channels (e.g., between four and eighteen conductive channels). According to a preferred embodiment, the cable comprises four electrically conductive channels. According to another preferred embodiment, the cable comprises six electrically conductive channels. According to a further preferred embodiment, the cable comprises twelve electrically conductive channels.

Each of the at least one electrically conductive channels is formed by at least one insulated conductor. The at least one insulated conductor is wound preferably spirally around the inner lumen of the cable. If the inner lumen is formed by an inner liner, the at least one insulated conductor is wound preferably spirally around the inner liner of the cable.

The at least one insulated conductor of the at least one electrically conductive channel comprises an electrical conductor and an insulating layer; preferably, the at least one electrically conductive channel consists of an electrical conductor and an insulating layer. Preferably, this applies to each of the electrically conductive channels of the cable.

The electrical conductor can be a single conductor or a plurality of individual conductors. Preferably, the electrical conductor is a metal wire or a plurality of metal wires, wherein the plurality of metal wires is wound, stranded, braided or rolled into a bundle of metal wires. The metal wire or the metal wire bundle can have a diameter in a range of 5 to 250 μm and preferably in a range of 10 to 120 μm. For example, the electrical conductor (e.g., the metal wire or the metal wire bundle) can have a diameter of about 85 μm.

The electrical conductor preferably consists of a metal selected from the group that consists of platinum, iridium, tantalum, palladium, titanium, iron, gold, molybdenum, niobium, tungsten, nickel, chromium, cobalt, stainless steel, nitinol, alloys of any of these metals and composite materials, of any of these metals. Stainless steel such as stainless steel AISI 316L, stainless steel AISI 301 or stainless steel AISI 304 is suitable as an electrical conductor. Platinum and platinum alloys such as Pt/Ir 10 or Pt/Ir 20 are suitable as electrical conductors. Nickel-cobalt alloys such as MP35N are suitable as electrical conductors.

The electrical conductor can also be provided with a coating (e.g., platinum) or plated to increase corrosion resistance. The electrical conductor can be, for example, a Pt-coated MP35N conductor or a Pt-coated tungsten-based conductor.

The insulating layer of the at least one electrical conductor preferably comprises a polymer that is selected from the group consisting of polyolefins (e.g., polyethylene), polyurethanes, polyimides, polyamides, polyaryletherketone (e.g., polyetheretherketone), fluorinated polymers (e.g., selected from the group of ethylenetetrafluoroethylene, polytetrafluoroethylene, perfluoroalkoxyalkanes, polyvinylidene fluorides, fluorinated ethylenepropylene, and mixtures thereof), and mixtures thereof.

Preferably, the insulating layer has a thickness in the range of 3 to 150 μm, and more preferably in the range of 5 to 40 μm. The insulating layer can, for example, have a thickness of about 15 μm.

The at least one electrically conductive channel is formed by at least one insulated conductor. The number of insulated conductors that form an electrically conductive channel can depend on the use of the lead or on the acceptable risk of failure of an insulated conductor.

For example, an electrically conductive channel can be formed by a single insulated conductor. This can have the advantage that the cable can contain a high number of conduction channels (e.g. 6, 8, 10, 12 or 18) since the single insulated conductor per conduction channel, which is arranged between the outer insulation and the inner lumen, takes up comparatively little space. According to a preferred embodiment of the present invention, the at least one electrically conductive channel is formed by a single insulated conductor.

On the other hand, an electrically conductive channel can also be formed by two or more insulated conductors. If an electrically conductive channel is formed by two or more insulated conductors, the electrical connection to the corresponding electrode, preferably a ring electrode, can be established with the two or more insulated conductors.

This can have the advantage that the contact stability of an electrode to the electrical conductors of an electrically conductive channel is higher since a larger contact area of the electrically conductive channel is available to establish the connection with the electrode. According to a further preferred embodiment of the present invention, each of the at least one electrically conductive channel is formed by two or more insulated conductors (e.g. two, three, four or five insulated conductors). According to one embodiment of the present invention, each of the at least one electrically conductive channel is formed by two or more insulated conductors that are arranged side by side.

According to a preferred embodiment of the present invention, the at least one electrically conductive channel, and preferably each electrically conductive channel, is formed by two or more insulated conductors which are preferably arranged side by side.

In method step b), a conductive paste is provided. A conductive paste in the sense of the present invention is a pasty composition, preferably a dispersion, which is curable, preferably under the influence of heat, and at least in the cured state enables an electrically conductive connection between at least two components. The conductive paste is preferably designed such that it creates a reliable, permanent electrically conductive connection between two or more components in a cured state.

The conductive paste can consist of a plurality of materials from inherently conductive substances such as inherently conductive polymers, such as poly-3,4-ethylenedioxythiophene (PEDOT), to curable media filled with conductive particulate fillers, to curable media provided with dissolved conductive substances.

Examples of conductive particles preferably comprise metal particles, in particular metal particles selected from the list consisting of platinum, iridium, tantalum, palladium, titanium, iron, gold, molybdenum, niobium, tungsten, nickel, chromium, cobalt, stainless steel, nitinol, rhodium, alloys of each of these metals and composites of each of these metals, or conductive carbon-containing compounds such as carbon nanotubes or graphene.

Curable media comprise, for example, polymers dissolved in volatile substances, preferably solvents, in particular organic solvents, which cure by evaporation of the solvent or comprise, for example, monomers which can cure by a polymerization reaction or mixtures thereof. The curable media can consist of monomers, or preferably the monomers are dissolved or dispersed in volatile substances, preferably a solvent, preferably an organic solvent.

Examples of the polymers preferably comprise polymers that are selected from the group consisting of silicones, polyolefins (e.g., polyethylene), polyether block amides (e.g., PEBAX®), polyurethanes, polyimides, polyamides, polyaryletherketones (e.g., polyether ether ketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof.

Examples of monomers that cure through a polymerization reaction are epoxy resins, bisphenol A diglycidyl ether and diethylenetriamines.

In further embodiments of the conductive paste, it comprises a curable medium which is produced by a mixture of at least two reactants. Upon coming into contact with each other, these reactants start a curing reaction which ultimately leads to an electrically conductive fixation of the electrode in method step h). The mixing of these reactants can be induced actively, for example by mixing a curable medium such as a two-component conductive adhesive, or can be induced passively, for example by curing the conductive paste through contact with water from the surrounding atmosphere. The latter conductive paste therefore comprises a curable medium like superglue.

The conductive paste, in particular the medium of the conductive paste, can comprise volatile substances, in particular volatile organic substances. Volatile substances are substances which evaporate during the curing of the conductive paste by means of heating. Examples of volatile substances are solvents, especially organic solvents.

In method step c), at least one contact opening is created near the distal end of the cable. The number of contact openings depends on the use of the lead. For example, for a lead having six electrodes at the distal end of the cable, preferably six disjoint contact openings are created in the proximity of the distal end of the cable so that one of the six electrodes can be selectively electrically connected to at least one of the corresponding electrically conductive channels by each of the six contact openings.

To create the contact openings, a part of the outer insulation of the cable as well as a part of the insulating layer of at least one of the electrically conductive channels running there is removed at the corresponding locations, so that at least one electrical conductor of an electrically conductive channel is accessible from the outside and can be electrically contacted at each of the contact openings. Preferably, only a radially outer part of the insulating layer of the electrical conductor is removed. At each of the contact openings, one of the provided electrodes can accordingly be selectively electrically connected to at least one of the electrically conductive channels over the further course of the method. In one embodiment, parts of the insulating layer of two electrically conductive channels are removed at the contact openings so that one of the electrodes can be selectively electrically connected to two electrically conductive channels via these contact openings.

Each of the contact openings comprises a contact opening cross-sectional area in a radial view of the contact opening. The contact opening cross-sectional area represents the cross-sectional area of the radially outer opening of the contact opening, wherein this can have any geometry such as round, oval, angular, rectangular, preferably square, or irregular. The side walls of the contact opening preferably run substantially perpendicular to a longitudinal axis of the cable, which can facilitate the creation of the contact opening, but can also include other angles. For example, the side walls of the contact opening can be shaped such that the cross-sectional area of the contact opening increases or decreases radially inward from the opening of the contact opening. An increasing cross-sectional area of the contact opening, for example, provides an undercut which can serve to improve the adhesion of the conductive paste or at least the hardened conductive paste in the contact opening. Each of the contact openings has a capacitance volume. The capacitance volume corresponds to the volume of the contact opening that can be filled with the conductive paste without the conductive paste protruding radially outwards from the contact opening. In the case of a contact opening with a capacity volume filled 100% with a conductive paste, the filled conductive paste is radially outer flush with the radially outer part of the outer insulation.

The contact opening can be created in different ways. For example, the contact opening can be created by cutting, punching or, preferably, by laser ablation.

In method step d), the contact opening is filled with the conductive paste in such a way that the filled volume of the conductive paste, the conductive paste volume, is greater than the capacity volume of the contact opening. In particular, the conductive paste volume corresponds to 105% to 200%, preferably 105% to 150%, more preferably 105% to 120%, of the capacity volume. This means that the conductive paste volume is between 5% and 100%, preferably between 5% and 50%, more preferably between 5% and 20%, larger than the capacity volume, based on the capacity volume. The 5% to 100% volume by which the conductive paste volume exceeds the capacitance volume results in a projection of conductive paste that protrudes radially from the contact opening. The contact opening is accordingly “overfilled” with conductive paste.

In method step e), at least one electrode for the lead is provided. The exact number of electrodes depends on the use of the lead. Preferably, a maximum of as many electrodes as electrically conductive channels are provided so that each of the electrodes can be electrically connected to an electrically conductive channel during the method. More preferably, the number of electrodes is half the number of electrically conductive channels provided so that over the course of the method, each of the electrodes can be electrically connected to two electrically conductive channels. In this way, each electrode comprises a second electrically conductive channel as a backup system in case the first electrically conductive channel fails.

Preferably, the electrodes are ring electrodes.

The electrodes can comprise a plurality of different materials or consist of different materials. Preferably, the electrodes comprise a metal that is selected from the group consisting of platinum, iridium, tantalum, palladium, titanium, iron, gold, molybdenum, niobium, tungsten, nickel, chromium, cobalt, steel, nitinol, alloys of any of these metals, and composites of any of these metals. Stainless steel is suitable as an electrode, for example stainless steel AISI 316L, stainless steel AISI 301 or stainless steel AISI 304. Platinum and platinum alloys such as Pt/Ir 10 or Pt/Ir 20 or nickel-cobalt alloys such as MP35N are also suitable as electrodes.

The choice of metal for the electrodes (and for the electrical conductors) may depend on the use of the lead according to the invention. For example, if the lead according to the invention is to be used in a permanent medical device, the electrodes may comprise, or in one embodiment consist of, platinum or a platinum-iridium alloy. If the lead according to the invention is to be used in a temporary medical device, the electrodes may comprise, and in one embodiment consist of, stainless steel. However, the application of the lead is not limited by the use of a particular metal.

The electrodes may also have a coating. Suitable coatings are metal nitrides such as TiN, metal oxides such as IrO₂ or conductive polymers. The surface of the electrodes can also be surface-structured, e.g., laser-structured.

Each of the electrodes, preferably ring electrodes, can have an outer diameter in the range of 200 to 5000 μm, in one embodiment in the range of 300 to 3000 μm and in one embodiment in the range of 500 to 1500 μm. Each of the electrodes may have a wall thickness in the range of 10 to 200 μm, in one embodiment 10 to 100 μm, and in one embodiment 30 to 70 μm. Furthermore, each of the electrodes may have a length in the range of 200 to 5000 μm, in one embodiment 300 to 3000 μm, and in one embodiment in the range of 500 to 1500 μm. According to one embodiment, each of the electrodes has an outer diameter in the range of 300 to 3000 μm and in one embodiment in the range of 500 to 1500 μm, a wall thickness in the range of 10 to 100 μm and in one embodiment of 30 to 70 μm, and a length in the range of 300 to 3000 μm and in one embodiment in the range of 500 to 1500 μm.

The electrodes have a contact surface with a contact surface cross-sectional area. The contact surface is the surface of the electrodes which, during the method, is brought into contact with the conductive paste, at least partially, in order to fix the electrode in an electrically conductive manner to the at least one electrically conductive channel. The contact surface cross-sectional area of the contact surface is larger than the contact opening cross-sectional area. If the contact surface is placed on the contact opening, the contact surface completely covers the contact opening and even protrudes beyond the edges of the contact opening.

In method step f), the at least one electrode is positioned at the position of the contact opening, wherein the contact surface is oriented in the direction of the contact opening. The contact surface accordingly faces the contact opening. In the case of a ring electrode as the electrode, the positioning of the electrode comprises, for example, axially drawing or pulling the ring electrode onto the cable until the ring electrode reaches the axial height of the contact opening.

In method step g), the positioned electrode is moved in the direction of the contact opening. At the latest, this leads to physical contact with the projection of conductive paste protruding from the contact opening, which is then moved laterally, or in other words “squeezed”, with progressive movement in the direction of the contact opening so that the projection of conductive paste is introduced between the outer insulation, which borders the contact opening, and at least a part of the contact surface, which projects beyond the contact opening. Since the contact surface comprises a larger cross-section than the contact opening, it projects beyond the contact opening so that the conductive paste is introduced, or in other words “squeezed”, into this part of the contact surface projecting beyond the contact opening during the movement.

Accordingly, the projection of conductive paste can provide improved mechanical fixation of the electrode, as well as improved electrical contact between the electrode and the electrically conductive channel of the cable, since at least the electrical contact between the electrode and the conductive paste can be improved. At the same time, however, no larger contact opening needs to be created, which reduces or even prevents the risk of electrical contact with an undesired electrically conductive channel since a larger contact opening can result in the removal of the insulating layer of an electrically conductive channel that should not be electrically contacted or should not be electrically contacted at this contact opening. Furthermore, the projection can reduce the negative effects of air inclusions between the conductive paste and the electrode since the projection leads to an increased contact area between the conductive paste and the electrode. Furthermore, the projection can lead to an improved sealing of at least one electrically conductive channel against moisture from outside the lead.

The curing of the conductive paste in method step h) during the method can, depending on the composition of the conductive paste, lead to shrinkage of the conductive paste, for example due to evaporation of volatile substances, in particular volatile organic substances, during curing. The projection of conductive paste can at least partially, preferably completely, compensate for this shrinkage, which can improve both the electrical contact between the cured conductive paste and the electrode as well as the mechanical connection between the cured conductive paste and the electrode.

In method step h), the conductive paste is cured so that the electrode is fixed in an electrically conductive manner to the electrically conductive channel via the cured conductive paste.

The type of curing depends on the type of conductive paste. For example, curing can be carried out by irradiating the conductive paste with electromagnetic radiation, which triggers a polymerization reaction in the conductive paste. Furthermore, curing can be carried out without active intervention, for example when a conductive paste such as a two-component conductive adhesive or superglue is used.

Curing preferably takes place by means of heat input, i.e. by heating the conductive paste, preferably because heating can be carried out particularly easily and/or such curable conductive pastes are usually easy to handle. For example, heating triggers a polymerization reaction in the conductive paste. Preferably, the heating causes volatile substances, such as one or more solvents of the conductive paste, to evaporate. The evaporation of these volatile substances ensures, for example, that the polymer previously dissolved in the curable medium of the conductive paste or the monomer previously dissolved in the curable medium solidifies through a polymerization reaction so that the conductive paste cures, and the electrode is fixed in an electrically conductive manner to the electrically conductive channel.

It should be noted that the described method can be carried out for each of the electrodes of the lead, wherein individual method steps can be carried out for each of the electrodes simultaneously or at different times. For example, it can be advantageous to make contact openings for each of the electrodes simultaneously, or at least directly following each other in time. For example, it is also possible to carry out the method for each of the electrodes one after the other and separately.

Furthermore, it should be noted that it can be advantageous to carry out method step d) in combination with method step h) in different ways. In one embodiment of the method, in method step d), the contact opening is filled with the claimed amount of conductive paste, and this is cured in method step h) in order to fix the electrode in an electrically conductive manner. In a further embodiment, in method step d) only some of the claimed amount of conductive paste is filled into the contact opening, for example only half of the final conductive paste volume, i.e. filling without a projection of conductive paste, wherein, after a curing step, this reduced amount of conductive paste is filled up with the remaining amount of conductive paste up to the claimed conductive paste volume, which then cures in method step h) in order to fix the electrode in an electrically conductive manner.

The conductive paste can have different conductive paste compositions.

A preferred embodiment of the method is characterized in that the conductive paste comprises a conductive paste composition which contains a proportion of a maximum of percent by weight of volatile substances, preferably volatile organic substances, based on the total mass of the conductive paste composition.

A volatile substance means a substance which escapes from the conductive paste composition during curing of the conductive paste under the influence of heat, preferably evaporates. Examples of volatile substances are solvents, especially organic solvents.

The conductive paste accordingly comprises volatile substances, preferably volatile organic substances, between 0 percent by weight and 5 percent by weight, based on the total mass of the conductive paste. As a result, preferably during curing of the conductive paste in method step h), there is only a slight or even substantially no change in volume, in particular shrinkage, of the conductive paste. This can improve both the electrical connection as well as preferably the mechanical connection between the electrode and the cured conductive paste.

It should be noted that the term “substances” refers to both a single volatile substance, for example a specific organic solvent, and a plurality of volatile substances, for example two or more, such as a mixture of two different organic solvents.

A preferred embodiment of the method is characterized in that the volatile organic substances are selected from the group consisting of solvents, preferably organic solvents, such as mono[(C12-14-alkyloxy)methyl] derivatives, ethyl acetate, tetrahydrofuran and neopentyl glycol diglycidyl ether.

Preferably, the conductive paste comprises conductive particles in a curable medium, wherein the medium comprises at least one polymer or monomer dissolved in volatile substances of the curable medium, in particular volatile organic substances of the medium. This type of conductive paste can be easily applied into the contact opening, for example by printing or dispensing, is easy to handle and can be cured, for example, by applying heat, which causes at least partial evaporation of the volatile substances and/or a polymerization reaction of the monomers. Furthermore, such a conductive paste can usually be easily processed mechanically, i.e. with as few manual steps as possible.

A preferred embodiment of the method is characterized in that the conductive paste composition contains a proportion of at least 0.5 percent by weight of volatile substances, preferably volatile organic substances, preferably solvents, in particular organic solvents, based on the total mass of the conductive paste composition.

Such a conductive paste can have improved application capabilities. In particular, the dispensing ability can be improved with such a conductive paste. Dispensing the conductive paste can be carried out particularly easily by machine, i.e. with as few manual method steps as possible.

A preferred embodiment of the method is characterized in that the conductive paste composition contains a proportion of 70 percent by weight to 90 percent by weight of conductive particles, in particular electrically conductive particles, preferably metallic electrically conductive particles, based on the total mass of the conductive paste composition. This ensures sufficiently good electrical conductivity even when the conductive paste is in a cured state.

The conductive particles can be of different nature. For example, the conductive particles can be conductive carbon compounds such as carbon nanotubes or graphene.

In the case of conductive carbon compounds, the conductive paste composition preferably contains a proportion of 2-25 percent by weight of conductive carbon particles, based on the total mass of the conductive paste composition.

A preferred embodiment of the method is characterized in that the conductive particles are metal particles. Metal particles usually have a high intrinsic electrical conductivity, are easy to process and can form a secure, stable and conductive connection between the electrode and the electrically conductive channel in the cured conductive paste, depending on the proportion of particles relative to the total mass of the cured conductive paste.

A preferred embodiment of the method is characterized in that the metal particles comprise a metal that is selected from the group consisting of platinum, iridium, tantalum, palladium, titanium, iron, gold, molybdenum, niobium, tungsten, nickel, chromium, cobalt, steel, nitinol and alloys comprising at least one of these metals. Metal particles can all comprise or consist of the same metal or can comprise mixtures of two or more of these metals or consist of such mixtures. Preferred metals are silver, platinum and gold.

A preferred embodiment of the method is characterized in that the conductive paste composition, in particular a curable medium of the conductive paste composition, comprises a polymer which is selected from the group consisting of polyimides and polyurethanes.

A preferred embodiment of the method is characterized in that the projection in the radial direction has a height in a range of 0.1 mm to 0.5 mm. If the projection in the radial direction is greater than 0.5 mm, the positioning of the electrode, in the form of a ring electrode, in method step f) is made more difficult since during positioning, a part of the conductive paste could be moved in an undesirable manner in the axial direction along the cable, in particular smeared. If the projection is smaller, during the movement of the electrode in method step g), too little conductive paste can be introduced between the outer insulation and part of the contact surface in order to achieve the desired technical effect.

A preferred embodiment of the method is characterized in that the curing in method step h) causes a radial contraction, i.e. a contraction in the radial direction relative to the cable, of the conductive paste, which corresponds to a maximum of 5% of a radial height of the contact opening. The conductive paste therefore preferably “shrinks” by no more than this 5% relative to the height of the contact opening, wherein this height substantially corresponds to the distance between the electrode and the section of the corresponding electrically conductive channel to be electrically contacted. Preferably, the conductive paste contracts in the radial direction during curing by no more than 4%, more preferably by no more than 3%, more preferably by no more than 1%. Most preferably, the conductive paste basically does not contract at all during curing.

This slight contraction of the conductive paste during curing can be influenced by the conductive paste composition. For example, conductive particles in the form of metal particles, preferably in a proportion of 70 percent by weight and 90 percent by weight, based on the total mass of the conductive paste, can ensure that the conductive paste contracts only slightly. Furthermore, only a small amount of volatile substances, such as only a small amount of organic solvents which evaporate when the conductive paste cures, can contribute to the small amount of shrinkage.

A preferred embodiment of the method is characterized in that the electrode is a ring electrode. Preferably, all electrodes are ring electrodes.

The movement in method step g) can be designed differently depending on the design of the electrode to be moved.

A preferred embodiment of the method, wherein the electrode is a ring electrode, is characterized in that the movement in method step g) comprises a radial compression of the ring electrode. Preferably, both the outer diameter as well as the inner diameter of the ring electrode are reduced substantially uniformly over the entire circumference of the ring electrode so that the outer diameter of the ring electrode at least approaches the outer diameter of the cable.

In the case of a ring electrode as the electrode, the contact surface of the electrode preferably corresponds to the radially inner ring surface of the ring electrode which is brought into contact with the conductive paste during the method.

The type of curing the conductive paste depends on the type and composition of the conductive paste. Preferably, the curing of the conductive paste occurs by applying heat.

A preferred embodiment of the method is characterized in that the curing in method step h) is carried out at a temperature which lies in a range between 110° C. and 140° C. On the one hand, this temperature range ensures that the remaining components of the lead experience as little thermal stress as possible during curing. Excessive thermal stress can, for example, lead to undesirable deformation of the lead, especially its polymer-based parts. Furthermore, this temperature range can cause the conductive paste to harden sufficiently quickly.

### FIGURES

The invention is further illustrated by way of example below by means of figures. The invention is not limited to the figures, in which:

FIG. 1 is an exemplary flow chart of a method for producing a bi- or multipolar lead for a medical device; and,

FIGS. 2a-f are exemplary intermediate products of the method for producing a multipolar lead in a schematic cross section.

### DETAILED DESCRIPTION OF THE INVENTION

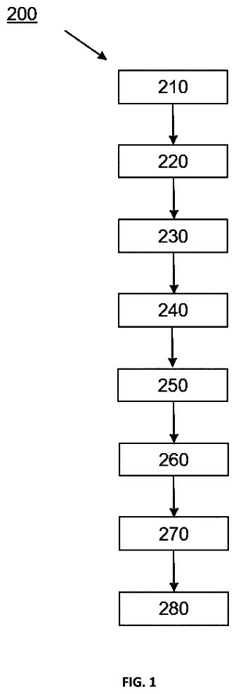

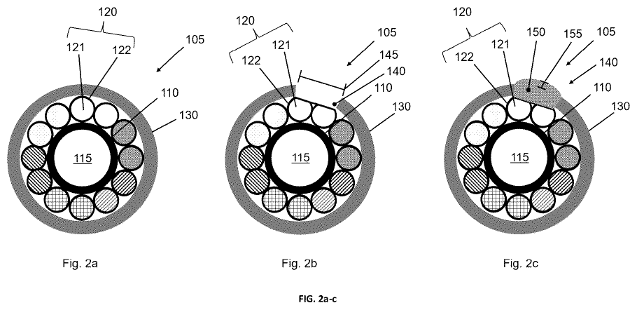

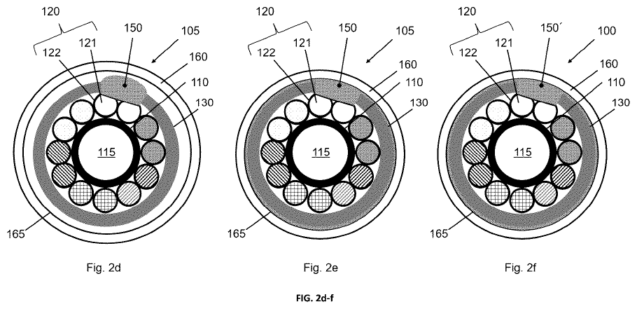

FIG. 1 is a flow chart of an exemplary method for producing a bi- or multipolar lead for a medical device with the method steps 210 to 280.

In a method step 210, a cable is provided. The cable comprises an outer insulation and an inner lumen, wherein the inner lumen is coaxially surrounded by the outer insulation and extends from a proximal end to a distal end of the cable. Furthermore, the cable comprises at least one electrically conductive channel. The number of electrically conductive channels influences the polarity of the lead. The at least one electrically conductive channel, preferably all electrically conductive channels of the lead, is formed by an insulated conductor which comprises an electrical conductor, such as a metal wire or a bundle of metal wires, and an insulating layer radially surrounding the electrical conductor, which surrounds the electrical conductor in a radially electrically insulating manner.

In a method step 220, a conductive paste is provided.

In a method step 230, at least one contact opening is created in the proximity of the distal end of the cable. The contact opening allows electrical contact of the at least one electrically conductive channel from outside the conductor at the position of the contact opening. For this purpose, the outer insulation as well as parts of the insulating layer of at least one electrically conductive channel are removed at the corresponding position of the cable. The contact opening is a kind of “hole” in the insulation lying radially above the electrical conductor of the electrically conductive channel. The contact opening has a contact opening cross sectional area. This represents the cross-sectional area of the “entrance” of the contact opening viewed radially from the outside.

In a method step 240, the contact opening is filled with the conductive paste. The type of filling depends on the type of conductive paste. Preferably, the filling comprises printing or dispensing the conductive paste into the contact opening. The contact opening is filled in such a way that a projection of conductive paste projects radially from the contact opening. For this purpose, the contact opening is filled with a conductive paste volume which corresponds to 105% to 200%, preferably 105% to 150%, more preferably 105% to 120%, of a capacity volume of the contact opening. The contact opening is accordingly “overfilled” with conductive paste.

In a method step 250, at least one electrode is provided. Preferably, the electrode is a ring electrode. The number of electrodes depends on the application of the lead to be produced. Preferably, the number of electrodes corresponds at most to the number of electrically conductive channels of the cable so that each of the electrically conductive channels can be selectively electrically contacted with one of the electrodes. In one embodiment of the method, the number of electrodes corresponds to half the number of electrically conductive channels of the cable so that each electrode can be selectively electrically contacted with two of the electrically conductive channels. This creates redundancy which can increase the reliability of the conductor.

The electrode has a contact surface which is brought into contact with the conductive paste during the method. The contact surface has a contact cross-sectional area which is larger than the contact opening cross-sectional area. In the manufactured conductor, the contact surface accordingly projects beyond the opening of the contact opening so that part of the contact surface projects beyond the contact opening.

In a method step 260, the electrode is positioned at the position of the contact opening. The contact surface of the electrode is oriented in the direction of the contact opening and the conductive paste located in the contact opening. In a preferred embodiment of the method, the electrode, preferably all electrodes, is a ring electrode, and the positioning thereof comprises axially drawing the electrode onto the cable up to the position of the corresponding contact opening.

In a method step 270, the positioned electrode is moved in the direction of the contact opening. At the latest, the electrode comes into contact with the projection of conductive paste so that, during the movement, it is introduced between the outer insulation and at least a part of the contact surface which projects beyond the contact opening. Preferably, the electrode is a ring electrode, and the projection of conductive paste is distributed substantially over the entire circumference of the inner surface of the ring during the movement.

In a method step 280, the conductive paste is cured in order to fix the electrode in an electrically conductive manner to the electrically conductive channel, in particular the electrical conductor of the electrically conductive channel. The type of curing depends on the type of conductive paste. Curing is preferably carried out by heating the conductive paste. Preferably, by heating the conductive paste, volatile substances, in particular volatile organic substances, evaporate from the conductive paste composition of the conductive paste. Furthermore, it is preferred that heating the conductive paste triggers a polymerization reaction of a monomer of the conductive paste composition, which cures the conductive paste.

FIGS. 2a-f are various exemplary intermediate products of the method 200 shown in FIG. 1 for producing a bi- or multipolar lead in a schematic cross section.

FIG. 2a is a cable 105 with an outer insulation 130 and an inner lumen 115, wherein the inner lumen 115 is arranged coaxially to the outer insulation 130 and is formed by an inner liner 110 in the form of a polymer tube. The inner lumen 115, and accordingly also the inner liner 110, extends axially from a proximal end of the cable 105 to a distal end of the cable 105 (not visible in the cross-section). The cable 105 comprises 12 electrically conductive channels 120 (provided with reference signs only as an example). In the shown embodiment, each of the electrically conductive channels 120 comprises exactly one insulated conductor which comprises an electrical conductor 121 with an insulating layer 122 surrounding the electrical conductor 121. The electrically conductive channels 120 are each shown in adjacent groups of two with different hatching, which is only intended to provide a better overview of the intermediate products shown. Otherwise, the electrically conductive channels 120 do not differ from each other. The electrically conductive channels 120 are wound spirally along the entire length of the inner liner 110 (not visible in the cross-section) and adjacent to one another around the inner lumen 115, in particular the inner liner 110.

FIG. 2b is the cable 105 from FIG. 2a, wherein a contact opening 140 was created in the proximity of the distal end of the cable 105 by removing parts of the outer insulation 130 and parts of the insulating layer 122 of two adjacent electrically conductive channels 120. By removing these electrically insulating components, selective electrical contact of the electrical conductors 121 of the corresponding electrically conductive channels 120 from outside the cable 105 is possible. In the shown embodiment, a contact opening 140 comprises exposing two adjacent electrical conductors 121 so that they can be electrically contacted with a single electrode. This increases the reliability of the final conductor in the event of a failure, for example due to severing, of one of the electrical conductors 121, in particular due to kinking. In this case, a current flow would still be possible via the second electrically conductive channel 120. The contact opening 140 comprises a radially outer opening with a contact opening cross-sectional area 145 (indicated by the drawn line).

FIG. 2c is the cable 105 from FIG. 2b, wherein the contact opening 140 has been filled with a previously provided conductive paste 150. The conductive paste 150 has accordingly been brought into contact with the two electrical conductors 121 of the two adjacent electrically conductive channels 120. In the shown embodiment, the conductive paste is a curable medium filled with silver particles, wherein the medium comprises 4 percent by weight of an organic solvent as a volatile substance and a monomer curable by means of a polymerization reaction, at least partially dissolved in the solvent. A filled conductive paste volume of the conductive paste 150 exceeds a capacity volume of the contact opening 140 so that a projection 155 of conductive paste 150 has formed above the contact opening. In the shown embodiment, the filled conductive paste volume is 115% of the capacity volume so that the projection 155 has a height of 0.2 mm in the radial direction.

FIG. 2d is the cable 105 from FIG. 2c, wherein a previously provided electrode 160 in the form of a ring electrode has been positioned at the contact opening 140 (cf. FIG. 2b or 2c). In the shown intermediate product of the method 100, the electrode 160 is still spaced from the cable 105. In particular, the electrode 160 was positioned such that a contact surface 165 of the electrode 160, in the shown embodiment the inner ring surface of the ring electrode, is oriented in the direction of the contact opening 140, and accordingly also in the direction of the conductive paste 150. The contact surface 165 has a contact cross-sectional area which is larger than the contact opening cross-sectional area 145 so that the contact surface 165 projects flat over the contact opening 140. In the cross-section, it is easily seen that the contact surface 165 extends around the full circumference of the cable 105. Furthermore, the ring electrode has a greater extension along the longitudinal axis of the cable 105 than the contact opening 140, which is not apparent in the shown cross-section.

FIG. 2e is the cable 105 from FIG. 2d, wherein the electrode 160 has been moved in the direction of the contact opening 140. In the shown embodiment, this movement comprises a radial compression of the ring electrode. The movement of the electrode 160 has resulted in the projection 155 (see FIG. 2c) of conductive paste 150 being introduced between the outer insulation 130 and the contact surface 165 which projects beyond the contact opening 140 (see FIG. 2b or 2c). In the shown embodiment, the projection 155 was distributed over the full circumference of the cable 105, in particular the full circumference of the outer insulation 130. In further embodiments (not shown), the projection 155 can also be spread out over a smaller area. The type of spreading of the projection 155 depends on its volume and the type of electrode 160.

FIG. 2f is a lead 100 made from the cable 105 from FIG. 2e by curing the conductive paste 150 by means of heating, whereby the conductive paste 150 was converted into a cured conductive paste 150′. This has fixed the electrode 160 in an electrically conductive manner to the two adjacent electrically conductive channels 120. The former projection 155 of conductive paste 150 (see, for example, FIG. 2c) achieved an improved electrically conductive fixation of the electrode 160.

The features disclosed in the claims, the description and the drawings may be essential for various embodiments of the claimed invention both individually and in any combination with one another.

### REFERENCE SIGNS

100 lead
105 cable
110 inner liner
115 inner lumen
120 electrically conductive channel
121 electrical conductor
122 insulating layer
130 outer insulation
140 contact opening
145 contact opening cross-sectional area
150 conductive paste
150′ cured conductive paste
155 projection
160 electrode
165 contact surface
200 method
210 providing cable
220 providing conductive paste
230 creating contact opening
240 filling
250 providing electrode
260 positioning
270 movement
280 curing

What is claimed is:

1. A method for producing a bi- or multipolar lead for a medical device, comprising the method steps of:
a) providing a cable, comprising
an outer insulation,
an inner lumen, wherein the inner lumen is arranged coaxially with respect to the outer insulation and extends in a longitudinal direction from a proximal end to a distal end of the cable, and
at least one electrically conductive channel, wherein the at least one electrically conductive channel is arranged between the outer insulation and the inner lumen of the cable, and, wherein the at least one electrically conductive channel is formed by at least one insulated conductor which comprises an electrical conductor and an insulating layer;
b) providing at least one conductive paste;
c) creating at least one contact opening in the proximity of the distal end of the cable, wherein, in order to create the contact opening, parts of the outer insulation are removed and parts of the insulating layer of at least one of the electrically conductive channels are removed so that the at least one electrically conductive channel can be selectively electrically contacted via the contact opening, wherein the contact opening comprises a contact opening cross-sectional area;
d) filling the contact opening with the conductive paste, wherein a filled conductive paste volume of the conductive paste comprises 105% to 200% of a capacity volume of the contact opening in order to create a projection of the conductive paste protruding from the contact opening;
e) providing at least one electrode, wherein the electrode comprises a contact surface with a contact cross-sectional area, wherein the contact cross-sectional area is larger than the contact opening cross-sectional area;
f) positioning the electrode at the position of the contact opening, wherein the contact surface is oriented in the direction of the contact opening;
g) moving the positioned electrode in the direction of the contact opening so that the projection of the conductive paste is introduced during the movement between the outer insulation and a part of the contact surface which projects beyond the contact opening;
h) curing the conductive paste to fix the electrode in an electrically conductive manner to the electrically conductive channel.

2. The method according to claim 1, wherein the conductive paste comprises a conductive paste composition which contains a proportion of a maximum of 5 percent by weight of volatile organic substances, based on the total mass of the conductive paste composition.

3. The method according to claim 2, wherein the volatile organic substances are selected from the group consisting of organic solvents.

4. The method according to claim 2, wherein the conductive paste composition contains a proportion of at least 0.5 percent by weight of volatile organic substances, based on the total mass of the conductive paste composition.

5. The method according to claim 2, wherein the conductive paste composition contains a proportion of 70-90 percent by weight of conductive particles.

6. The method according to claim 5, wherein the conductive particles are metal particles.

7. The method according to claim 6, wherein the metal particles comprise a metal which is selected from the group consisting of platinum, iridium, tantalum, palladium, titanium, iron, gold, molybdenum, niobium, tungsten, nickel, chromium, cobalt, stainless steel, nitinol, alloys of any of these metals, and composites of any of these metals.

8. The method according to claim 2, wherein the conductive paste composition comprises a polymer which is selected from the group consisting of polyimides and polyurethanes.

9. The method according to claim 1, wherein the projection in the radial direction has a height in a range of 0.1 mm to 0.5 mm.

10. The method according to claim 1, wherein the curing in method step h) causes a radial contraction of the conductive paste which corresponds to a maximum of 5% of a radial height of the contact opening.

11. The method according to claim 1, wherein the electrode is a ring electrode.

12. The method according to claim 11, wherein the movement in method step g) comprises a radial compression of the ring electrode.

13. The method according to claim 11, wherein the positioning in method step f) comprises drawing the ring electrode onto the cable.

14. The method according to any of claim 11, wherein the contact surface is a radially inner ring inner surface of the ring electrode.

15. The method according to claim 1, wherein the curing in method step h) is carried out at a temperature which lies in a range between 110° C. and 140° C.

16. The method according to claim 3, wherein the conductive paste composition contains a proportion of at least 0.5 percent by weight of volatile organic substances, based on the total mass of the conductive paste composition.

17. The method according to claim 3, wherein the conductive paste composition contains a proportion of 70-90 percent by weight of conductive particles.

18. The method according to claim 4, wherein the conductive paste composition contains a proportion of 70-90 percent by weight of conductive particles.

19. The method according to claim 12, wherein the positioning in method step f) comprises drawing the ring electrode onto the cable.
